(19) United States
(12) Patent Application Publication
(10) Pub. No.: US 2012/0141420 A1
(43) Pub. Date: Jun. 7, 2012

(54) TARGETED DELIVERY OF G-CSF FOR THE TREATMENT OF AMYOTROPHIC LATERAL SCLEROSIS

(75) Inventors: Armin SCHNEIDER, Heidelberg (DE); Alexandre HENRIQUES, Strasbourg (FR); Claudia PITZER, Rauenberg (DE)

(73) Assignee: SYGNIS BIOSCIENCE GmbH & Co. KG, Heidelberg (DE)

(21) Appl. No.: 13/307,308

(22) Filed: Nov. 30, 2011

Related U.S. Application Data
(60) Provisional application No. 61/417,986, filed on Nov. 30, 2010.

(51) Int. Cl. A61K 35/76; A61P 25/28

(52) U.S. Cl. 424/93.2

(57) ABSTRACT

The present invention relates to a method of treating Amyotrophic Lateral Sclerosis by the targeted delivery of granulocyte-colony stimulating factor to the central nervous system with an adeno-associated virus (AAV) vector.

### CROSS-REFERENCE TO RELATED APPLICATIONS

This application claims the benefit of U.S. provisional application Ser. No. 61/417,986 filed Nov. 30, 2010.

### FIELD OF THE INVENTION

The present invention relates to a method of treating Amyotrophic Lateral Sclerosis by the targeted delivery of granulocyte-colony stimulating factor to the central nervous system with an adeno-associated virus (AAV) vector.

### BACKGROUND OF THE INVENTION

Amyotrophic Lateral Sclerosis (ALS) is an incurable fatal motoneuron disease, characterized by progressive weakness, muscle wasting and death ensuing 3-5 years after diagnosis (Mitchell et al., (2007), Lancet 369: 2031-2041). The etiopathogenesis and pathophysiology of ALS is complex with many players involved that lead to the functional decline of the motor pathway (Gonzalez de Aguilar et al., (2007), J Neurochem 101: 1153-1160; Pasinelli et al., (2006), Nat Rev Neurosci 7: 710-723; Rothstein J D, (2009), Ann Neurol 65 Suppl 1: S3-9). Due to insufficient insights into the molecular pathway(s) crucial to ALS pathogenesis the most rational strategy at present remains to rescue and strengthen motor units with neurotrophic factors (Henriques et al., (2010), Front Neurosci 4: 32). A number of growth factors have been clinically tested in ALS without success so far, but major pharmacokinetic problems and unexpected peripheral effects preclude any conclusion as to the true therapeutic potential of this concept (Henriques et al., (2010), Front Neurosci 4: 32).

One challenge for growth factor treatment in ALS is the likely need for a very chronic treatment with those proteins. In the case of G-CSF, any form of systemic delivery, such as subcutaneous delivery, has the inherent consequence of a chronic rise in white blood cells (WBC). The effects of such chronic elevation of WBCs have not been explored extensively in humans. In addition, the limited plasma half-life (˜4 hrs) would require repeated dosing or some sort of pump delivery of the protein.

To circumvent these problems, direct delivery of G-CSF to the central nervous system (CNS) using viral vectors might be advantageous. Recombinant adeno-associated virus (Atchison et al., (1965), Science 149: 754-756) is replication-deficient, derived from a non-pathogenic virus, and is able to infect numerous cell types, including neurons, resulting in its presence in the nucleus as episomal concatamers (Bouard et. al., (2009), Br J Pharmacol 157: 153-165). In non-dividing neuronal cells, the virus may persist in that form for the lifetime of the cell. AAV serotype 2 was described to be able to transduce spinal motoneurons of SOD-1 (G93A) mice after intramuscular and intraspinal injections (Dodge et al., (2008), Mol Ther 16: 1056-1064; Kaspar et. al., (2003), Science 301: 839-842), and lead to the production of neurotrophic factors.

### SUMMARY OF THE INVENTION

Using a recombinant AAV virus to deliver G-CSF to spinal motor neurons by either intramuscular or direct intraspinal injection of AAV, the present inventors demonstrate that intraspinal delivery improved efficacy of G-CSF treatment and decreased peripheral load and elevation of leukocyte count.

The present invention provides a method of treating amyotrophic lateral sclerosis (ALS) by delivering to a spinal cord region of the mammalian subject, a recombinant vector comprising at least two adeno-associated virus (AAV) inverted terminal repeats (ITRs) flanking a polynucleotide encoding mammalian G-CSF operably linked to a transcriptional promoter that can express the polynucleotide.

The present invention also provides using various forms of the AAV virus in the recombinant delivery vehicle as having different portions from various serotypes of AAV to improve the transduction efficiency, e.g., AAV-1 and AAV-2.

In one embodiment, the spinal cord region to which delivery is effectuate is a lumbar region or a cervical region.

### BRIEF DESCRIPTION OF THE DRAWINGS

FIG. 1G-CSF expression in motoneurons after intramuscular and intraspinal injection of AAV. (A, C) Following intramuscular injection no virus expression can be detected in the spinal cord. However, the virus expresses eGFP in the skeletal muscle (E). In contrast, intraspinal injection leads to a strong transduction of motoneurons (B, D). As expected, no virus expression is seen in the musculature (F).

FIG. 2 Distribution of G-CSF after intramuscular and intraspinal injection of AAV vector. (A) The level of G-CSF in the serum is increased in mice injected with AAV G-CSF compared to mice injected with the control vector (n=10, *p<0.05). Muscular injection of AAV G-CSF leads to a higher level of circulating G-CSF compared to the spinal injection (p<0.05). (B) Level of G-CSF in the total spinal cord is increased in mice injected with AAV G-CSF compared to mice injected with the control vector (n=6, p<0.05). The increase following intraspinal injection is 150-fold higher than after intramuscular injection (p<0.05). (C) Neutrophil numbers following intramuscular and intraspinal injections of AAV G-CSF. The number of neutrophils increases after both muscular and spinal injections. Neutrophil counting was performed using an automatic counting system (n=7, *p<0.05).

FIG. 3 AAV G-CSF improves motor functions of SOD-1 (G93A) mice. (A) Muscular strength measured by the grip strength test. AAV G-CSF treatment leads to a relative preservation of the muscular strength of SOD-1 (G93A) mice at the mid- to end-point of the disease (n=12). (B) Performance on Rotarod. AAV G-CSF treatment leads to an improvement of endurance/coordination performance at the mid- to end-point of the disease (n=12).

FIG. 4 AAV G-CSF delays symptoms progression and enhances survival of SOD-1 (G93A) mice. Kaplan-Meyer graphs showing time to body mass decrease (A), onset of paresis (B) and clinical endstage of the disease (C). (A) Body mass decrease, associated with muscular atrophy, is delayed after AAV G-CSF treatment (p<0.05). (B) First manifestation of paresis of the hind limbs is delayed after AAV G-CSF treatment (p<0.05). (C) Survival of SOD-1 (G93A) mice is increased by 10% after AAV G-CSF treatment (p<0.05).

FIG. 5 AAV G-CSF improves α-motoneuron survival in SOD-1 (G93A) mice. (A, C) Quantification of surviving α-motoneurons after AAV G-CSF treatment, in SOD-1 (G93A) mice at 15 weeks of age. Motoneuron survival is increased after spinal injection of AAV G-CSF at both the cervical (A, +50%; p<0.05; n=9) and lumbar (C, +35%; p<0.05; n=9) level of the spinal cord when compared to control. (B, D) Size evaluation of all ChAT+ cells in SOD-1 (G93A) mice at 15 weeks of age. There is an upward shift in mean size distribution by AAV G-CSF treatment at both the cervical (B) and lumbar (D) level (p<0.05).

FIG. 6 AAV G-CSF enhances muscular innervation in SOD-1 (G93A) mice. (A) Double fluorescence immunostaining of nicotinic acetylcholine receptors, (ACh r) and axons (neurofilament-L) in the gastrocnemius muscle of 15 week old mice (AAV eGFP, AAV G-CSF and wild type mice). (B) Innervated NMJs in percentage of total NMJs, in the gastrocnemius muscle of 15 week old mice. AAV G-CSF treatment increases the fraction of innervated sites (p<0.0005; n=5). (C) Number of innervated NMJs normalized to muscle volume. AAV G-CSF treatment increases the number of innervated NMJs per volume (p<0.0005; n=5).

FIG. 7 AAV G-CSF enhances reinnervation after sciatic nerve crush in SOD-1 (G93A) mice. Innervated NMJs in percentage of total NMJs, in the gastrocnemius muscle of 15 week old mice, 6 days after sciatic nerve crush injury. AAV G-CSF treatment increases reinnervation (p<0.005; n=4).

FIG. 8 AAV gene in the spinal cord after intramuscular injection. RT-PCR for viral genome in the lumbar spinal cord of SOD-1 (G93A) mice after intramuscular injection of AAV G-CSF. (+), AAV plasmid.

FIG. 9 Complete recovery of mice after intraspinal injection of AAV. (A) One week after surgery, motor functions of operated mice are similar to their previous level for the overall performance on rotarod test (p=0.80; n=12) and for the muscle strength measured with the grip strength test (p=0.25; n=12). (B) One week after surgery, body mass of operated mice is maintained and shows a trend for increase (p<0.12; n=12).

FIG. 10 Intramuscular injection of AAV G-CSF enhances survival of SOD-1 (G93A) mice. Kaplan-Meyer graphs showing time to clinical endstage of the disease. Survival of SOD-1 (G93A) mice is increased by 7% after intramuscular AAV G-CSF treatment (p<0.05, n=12).

FIG. 11 Size distribution of ChAT-positive cells in the spinal cord. Shown is the histogram distribution of ChAT-positive cells in C3/C4 and L3/L4 spinal segments for the SOD-1 (G93A) mice treated with G-CSF, the SOD-1 (G93A) mice injected with the control vector and the C57 B/6 wild type mice.

FIG. 12 Microglia cells in the spinal cord of SOD-1 (G93A) mice. Quantification of microglia number in the lumbar spinal of SOD-1 (G93A) and wild type mice at 15 weeks of age. G-CSF has no influence on the number of microglia in the spinal cord of SOD-1 (G93A) mice when compared to the control group of SOD-1 (G93A) mice (p<0.29, n=4). SOD-1 (G93A) mice have however more microglia in the spinal cord when compared to wild type (p<0.05, n=4).

FIG. 13 shows an alignment of the GCSF from different species using the ClustalW algorithm (SEQ ID NOS:1-6) (MEGALIGN™, Lasergene, Wisconsin).

### DETAILED DESCRIPTION OF THE INVENTION

Granulocyte-colony stimulating factor (G-CSF) is a well known growth factor. The G-CSF that can be employed in the inventive methods described herein are those full length coding sequences, protein sequences, and the various functional variants, muteins, and mimetics that are known and available. In the discussion that follows these are referred to as G-CSF derivatives.

G-CSF stimulates proliferation, survival, and maturation of cells committed to the neutrophilic granulocyte lineage through binding to the specific G-CSF receptor (G-CSFR) (see Hartung T., et al., Curr. Opin. Hematol. 1998; 5:221-5). G-CSFR mediated signaling activates the family of Signal Transducer and Activator of Transcription (STAT) proteins which translocate to the nucleus and regulate transcription (Darnell J E Jr., Science 1997; 277:1630-5). G-CSF is typically used for the treatment of different kinds of neutropenia in humans. It is one of the few growth factors approved for clinical use. In particular, it is used to reduce chemotherapy (CT)-induced cytopenia (Viens et al., J. of Clin. Oncology, Vol. 20, No. 1, 2002:24-36). G-CSF has also been implicated for therapeutic use in infectious diseases as potential adjunctive agent (Hübel et al., J. of Infectious Diseases, Vol. 185:1490-501, 2002). G-CSF has reportedly been crystallized to some extent (EP 344 796), and the overall structure of G-CSF has been surmised, but only on a gross level (Bazan, Immunology Today 11: 350-354 (1990); Parry et al. J. Molecular Recognition 8: 107-110 (1988)).

Neurotrophic properties of G-CSF. It has been shown that G-CSF and its receptor are expressed by neurons in many regions of the adult brain and spinal cord (Schneider et al., (2005), J Clin Invest 115: 2083-2098; Pitzer et al., (2008) Brain 131: 3335-3347; Pitzer et al., (2010) J Neurochem). G-CSF appears clinically attractive, since it is generally well-tolerated, crosses the intact blood-brain barrier (BBB) (Schneider et al., (2005), J Clin Invest 115: 2083-2098) and its pharmacokinetic properties are well established. A large number of studies in various animal models of neurodegenerative diseases demonstrated that G-CSF is neuroprotective and pro-regenerative in models of stroke (Schneider et al., (2005), J Clin Invest 115: 2083-2098) of Parkinson's disease (Meuer et al., (2006), J Neurochem 97: 675-686), and of spinal cord injury (Pitzer et al., (2010) J Neurochem).

In 2008, it was reported that G-CSF had beneficial effects in SOD-1 (G93A) transgenic mice, an animal model for ALS, after systemic delivery using subcutaneous pump-delivery or transgenic expression of G-CSF (Pitzer et al., (2008) Brain 131: 3335-3347). One key mechanism of G-CSF protection is its direct anti-apoptotic effect on motoneurons, as supported by G-CSF-mediated protection of motoneurons after neonatal sciatic nerve axotomy (Henriques et al., (2010), BMC Neurosci 11: 25).

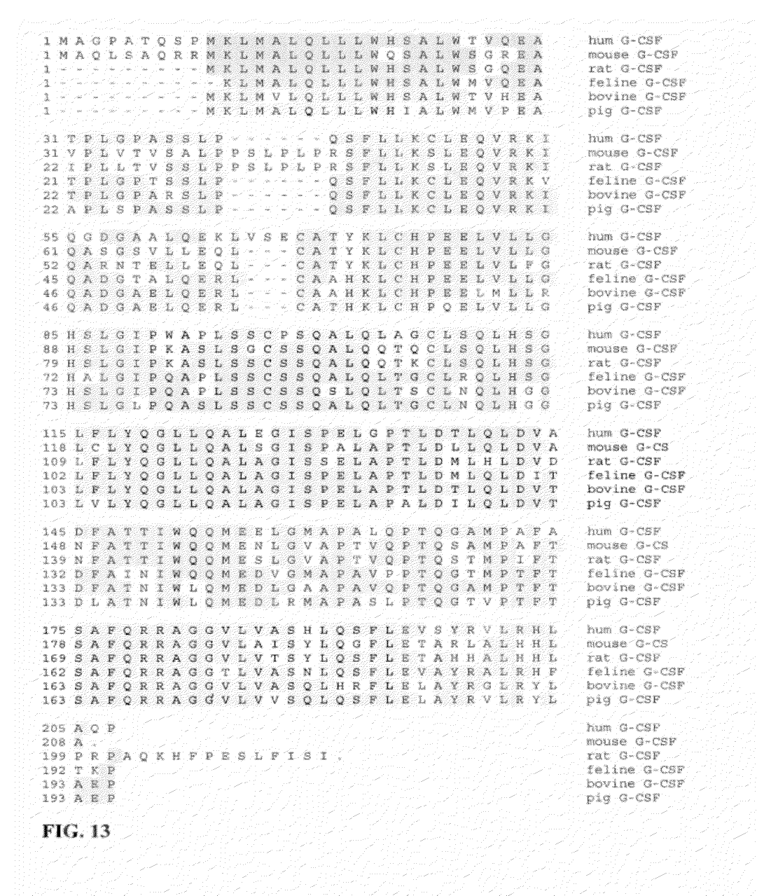

FIG. 13 shows an alignment of the G-CSF from different species using the ClustalW algorithm (SEQ ID NOS:1-6) (MEGALIGN™, Lasergene, Wisconsin).

The structure of both the coding DNA and protein are known as well as methods for recombinantly producing mammalian pluripotent granulocyte colony-stimulating factor (WO 87/01132; U.S. Pat. No. 4,810,643). For example, several amino acid sequences corresponding to G-CSF is shown in FIG. 13 and the Sequence Listing, i.e., SEQ ID NOS: 1, 2, 3, 4, 5, and 6.

In one embodiment, the proteins that are at least 70%, preferably at least 80%, more preferably at least 90% identical to the full-length human G-CSF amino acid sequences described herein can be employed in the present invention, e.g., SEQ ID NO:1. In another embodiment, the G-CSF that can be used are those that are encoded by polynucleotide sequence with at least 70%, preferably 80%, more preferably at least 90%, 95%, and 97% identity to the wildtype full-length human G-CSF coding sequence, e.g., a polynucleotide encoding SEQ ID NO:1, these polynucleotides will hybridize under stringent conditions to the coding polynucleotide sequence of the wild-type full length human G-CSF. The terms “stringent conditions” or “stringent hybridization conditions” includes reference to conditions under which a polynucleotide will hybridize to its target sequence, to a detectably greater degree than other sequences (e.g., at least 2-fold over background). Stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides), for example, high stringency conditions include hybridization in 50% formamide, 1 M NaCl, 1% SDS at 37° C., and a wash in 0.1×SSC at 60 to 65° C. (see Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995)). Amino acid and polynucleotide identity, homology and/or similarity can be determined using the ClustalW algorithm, MEGALIGN™, Lasergene, Wisconsin), WU-Blast, NCBI-Blast and/or FASTA.

Examples of the various G-CSF functional variants, muteins, and mimetics include functional fragments and variants (e.g., structurally and biologically similar to the wild-type protein and having at least one biologically equivalent domain), chemical derivatives of G-CSF (e.g., containing additional chemical moieties, such as polyethyleneglycol and polyethyleneglycol derivatives thereof, and/or glycosylated forms such as Lenogastrim™), and peptidomimetics of G-CSF (e.g., a low molecular weight compound that mimics a peptide in structure and/or function (see, e.g., Abell, Advances in Amino Acid Mimetics and Peptidomimetics, London: JAI Press (1997); Gante, Peptidmimetica—massgeschneiderte Enzyminhibitoren Angew. Chem. 106: 1780-1802 (1994); and Olson et al., J. Med. Chem. 36: 3039-3049 (1993)).

Additional examples of G-CSF derivatives include a fusion protein of albumin and G-CSF (Albugranin™), or other fusion modifications such as those disclosed in U.S. Pat. No. 6,261,250); PEG-G-CSF conjugates and other PEGylated forms; those described in WO 00/44785 and Viens et al., J. of Clin. Oncology, Vl., Nr. 1, 2002: 24-36; norleucine analogues of G-CSF, those described in U.S. Pat. No. 5,599,690; G-CSF mimetics, such as those described in WO 99/61445, WO 99/61446, and Tian et al., Science, Vol. 281, 1998:257-259; G-CSF muteins, where single or multiple amino acids have been modified, deleted or inserted, as described in U.S. Pat. Nos. 5,214,132 and 5,218,092; those G-CSF derivatives described in U.S. Pat. No. 6,261,550 and U.S. Pat. No. 4,810,643; and chimeric molecules, which contain the full sequence or a portion of G-CSF in combination with other sequence fragments, e.g. Leridistim—see, for example, Streeter, et al. (2001) Exp. Hematol., 29, 41-50, Monahan, et al. (2001) Exp. Hematol., 29, 416-24, Hood, et al. (2001) Biochemistry, 40, 13598-606, Farese et al. (2001) Stem Cells, 19, 514-21, Farese, et al. (2001) Stem Cells, 19, 522-33, MacVittie, et al. (2000) Blood, 95, 837-45. Additionally, the G-CSF derivatives include those with the cysteines at positions 17, 36, 42, 64, and 74 (of the 174 amino acid species (SEQ ID NO:7) or of those having 175 amino acids, the additional amino acid being an N-terminal methionine (SEQ ID NO:8) substituted with another amino acid, (such as serine) as described in U.S. Pat. No. 6,004,548, G-CSF with an alanine in the first (N-terminal) position; the modification of at least one amino group in a polypeptide having G-CSF activity as described in EP 0 335 423; G-CSF derivatives having an amino acid substituted or deleted in the N-terminal region of the protein as described in EP 0 272 703; derivatives of naturally occurring G-CSF having at least one of the biological properties of naturally occurring G-CSF and a solution stability of at least 35% at 5 mg/ml in which the derivative has at least Cys¹⁷ of the native sequence replaced by a Ser¹⁷ residue and Asp²⁷ of the native sequence replaced by a Ser²⁷ residue as described in EP 0 459 630; a modified DNA sequence encoding G-CSF where the N-terminus is modified for enhanced expression of protein in recombinant host cells, without changing the amino acid sequence of the protein as described in EP 0 459 630; a G-CSF which is modified by inactivating at least one yeast KEX2 protease processing site for increased yield in recombinant production using yeast as described in EP 0 243 153; lysine altered proteins as described in U.S. Pat. No. 4,904,584; cysteine altered variants of proteins as described in WO/9012874 (U.S. Pat. No. 5,166,322); the addition of amino acids to either terminus of a G-CSF molecule for the purpose of aiding in the folding of the molecule, for example after prokaryotic expression as described in AU-A-10948/92; substituting the sequence Leu-Gly-His-Ser-Leu-Gly-Ile (SEQ ID NO:9) at position 50-56 of G-CSF with 174 amino acids (SEQ ID NO:7), and position 53 to 59 of the G-CSF with 177 amino acids, or/and at least one of the four histadine residues at positions 43, 79, 156 and 170 of the mature G-CSF with 174 amino acids (SEQ ID NO:7) or at positions 46, 82, 159, or 173 of the mature G-CSF with 177 amino acids as described in AU-A-76380/91; and a synthetic G-CSF-encoding nucleic acid sequence incorporating restriction sites to facilitate the cassette mutagenesis of selected regions and flanking restriction sites to facilitate the incorporation of the gene into a desired expression system as described in GB 2 213 821. Further examples of G-CSF are described in U.S. Pat. Nos. 6,632,426 and 7,884,069. The contents of the above are incorporated herein by reference.

The various functional derivatives, variants, muteins and/or mimetics of G-CSF preferably retain at least 20%, preferably 50%, more preferably at least 75% and/or most preferably at least 90% of the biological activity of wild-type mammalian G-CSF activity—the amount of biological activity include 25%, 30%, 35%, 40%, 45%, 55%, 60%, 65%, 70%, 75%, 80%, 85%, 95%; and all values and subranges there between. Furthermore, the functional derivatives, variants, muteins and/or mimetics of G-CSF can also have 100% or more of the biological activity relative to wild-type mammalian G-CSF activity—the amount of biological activity including at least 105%, at least 110%, at least 125%, at least 150%, and at least 200%.

To measure the biological activity of G-CSF, several known assays can be employed singularly or in combination. One example of determining G-CSF function is illustrated in Example 1 of U.S. Pat. No. 7,884,069. Other methods for determining G-CSF function are known and include a colony formation assay employing murine bone marrow cells; stimulation of proliferation of bone marrow cells induced by G-CSF; specific bioassays with cells lines that depend on G-CSF for growth or that respond to G-CSF (e.g., AML-193; 32D; BaF3; GNFS-60; HL-60, M1; NFS-60; OCI/AML1a; and WEHI-3B). These and other assays are described in Braman et al. Am. J. Hematology 39: 194-201 (1992); Clogston C L et al Anal Biochem 202: 375-83 (1992); Hattori K et al Blood 75: 1228-33 (1990); Kuwabara T et al Journal of Pharmacobiodyn 15: 121-9 (1992); Motojima H et al Journal of Immunological Methods 118: 187-92 (1989); Sallerfors B and Olofsson European Journal of Haematology 49: 199-207 (1992); Shorter S C et al Immunology 75: 468-74 (1992); Tanaka H and Kaneko Journal of Pharmacobiodyn. 15: 359-66 (1992); Tie F et al Journal of Immunological Methods 149: 115-20 (1992); Watanabe M et al Anal. Biochem. 195: 38-44 (1991).

In one embodiment, the G-CSF is modified or formulated, or is present as a G-CSF mimetic that increases its ability to cross the blood-brain barrier, or shift its distribution coefficient towards brain tissue. An example of such a modification is the addition of PTD or TAT sequences (Cao et al. (2002) J. Neurosci. 22:5423-5431; Mi et al. (2000) Mol. Ther. 2:339-347; Morris et al. (2001) Nat Biotechnol 19:1173-1176; Park et al. (2002) J Gen Virol 83:1173-1181). These sequences can also be used in mutated forms, and added with additional amino acids at the amino- or carboxy-terminus of proteins. Also, adding bradykinin, or analogous substances to an intravenous application of any G-CSF preparation will support its delivery to the brain, or spinal cord (Emerich et al. (2001) Clin Pharmacokinet 40:105-123; Siegal et al (2002) Clin Pharmacokinet 41:171-186).

In one embodiment the biological activity of G-CSF is enhanced by fusion to another hematopoietic factor. The enhanced activity can be measured in a biological activity assay as described above. Such a preferred modification or formulation of G-CSF leads to an increased antiapoptotic effect and/or an increase in neurogenesis. An example for such a modification is Myelopoietin-1, a G-CSF/IL-3 fusion protein (McCubrey et al. (2001), Leukemia, 15, 1203-16) or Progenipoietin-1 (ProGP-1) is a fusion protein that binds to the human fetal liver tyrosine kinase flt-3 and the G-CSF receptor.GM-CSF

G-CSF and derivatives thereof are provided to the individual by administrating one or more nucleic acids that encodes these factors in the form of a recombinant AAV vector. The isolated and purified nucleic acid encoding and expressing the protein or polypeptide is operably linked to a promoter that is suitable for expression in neural cells. and/or exclusively expressed in neuronal cells, in particular in motoneurons.

Adeno-associated virus is a human parvovirus with a single-stranded DNA genome of 4.7 kb AAV, a non-pathogenic, helper-dependent virus, is an attractive vector for gene therapy as it exhibits a wide host and tissue range and is able to replicate in cells from any species as long as there is a successful infection of such cells with a suitable helper virus [e.g., Adenovirus (Ad) or Herpesvirus]. The host and tissue tropism of AAV is determined by the ability of its capsid to bind to specific cellular receptors and/or co-receptors. The non-pathogenic nature and the ability to integrate itself into a variety of tissues has established this virus and vectors that are obtained from this virus as a gene delivery and expression vehicle in various mammalian cells and tissues.

AAV of several different serotypes are known, e.g., AAV 1 and AAV 2. AAV contains two open reading frames (ORFs), Rep and Cap, which are flanked by two inverted terminal repeats (ITRs). The ITRs are 160 nucleotides in length and are considered to be the only cis elements required for replication and site-specific integration of the AAV genome. In addition to the ITR elements, the Rep gene is necessary in trans to target the integration event to the AAVS1 site located on human chromosome 19. However, that the ITRs are the only required structural feature needed for integration into a variety of chromosomal locations and coupled with an adjoining promoter finds utility in expression various genes in a variety of tissues, such as eye, nervous system, muscle, lung amongst others.

In one embodiment, the expression construct that includes the promoter and G-CSF encoding sequence replaces all or a part of the AAV Rep and/or Cap coding sequences and in one preferred aspect, the only sequences of AAV remaining are the sequences of the ITRs flanking the remaining recombinant construct, e.g., the promoter and G-CSF gene are minimally present and in some embodiments the only viral sequences in the delivery vehicle. Multiple copies of the expression construct of the promoter and the G-CSF encoding sequence can be provided as long as the recombinant construct can be packaged within the viral capsid molecule.

The sequences of the various serotypes of AAV are well known as well as the 145 base pair ITRs that flank the viral genome (see, e.g., Srivastava et al (1983) J Virol. 45 (2): 555-564.

Of the various AAV serotypes, serotype 2 AAV has been the most extensively studied and characterized. Accordingly, serotype 2 rAAV vectors (i.e., nucleic acid constructs) and virions (i.e., encapsidated vectors) have been used as the vector of choice for gene transfer protocols. Animal experiments, however, have shown that dramatic differences exist in the transduction efficiency and cell specificity of rAAV virions of different serotypes (Chao et al., Mol. Ther. 2:619 623, 2000; Davidson et al., PNAS 97:3428 3432, 2000; and Rabinowitz et al., J. Virol. 76:791 801, 2002). Accordingly, in one embodiment, pseudotyped AAV vectors, hybrids of various serotypes can be used, e.g., as disclosed in U.S. Pat. No. 7,094,604. In one embodiment, the pseudoytped AAV vector includes portions of AAV-1 and AAV-2.

Methods of manufacturing a recombinant AAV expression vector including the G-CSF encoding sequence(s) is well-known to the skilled person that can include helper AAV virus to provide in trans the AAV structural and non-structural proteins with a further helper virus, e.g., adenovirus or herpesvirus and also can be accomplished in an in vitro system in a cell free extract (e.g., U.S. Pat. No. 5,741,683).

Suitable promoters for operable linkage to the isolated and purified nucleic acid are known in the art. For example, the isolated and purified nucleic acid encoding the polypeptide is operably linked to a promoter selected from the group consisting of the muscle creatine kinase (MCK) promoter (Jaynes et al., Mol. Cell Biol. 6: 2855-2864 (1986)), the cytomegalovirus (CMV) promoter, a tetracycline/doxycycline-regulatable promoter (Gossen et al., PNAS USA 89: 5547-5551 (1992)) Further suitable promotors are those ubiquitous expressed throughout neuronal tissue like the neuron specific enolase promoter (NSE), the Cam Kinase II promoter (CamKII), the Synapsin I promoter, the prion protein promoter (PrP) and the beta 3 tubulin promoter (Tubb3) or those known to be expressed in motoneurons like the vesicular acetylcholine transporter promoter (VAChT) and the choline acetyl transferase promoter (Chat).

Generally, to ensure effective transfer of the vectors of the present invention, about 1 to about 5,000 copies of the vector are employed per cell to be contacted, based on an approximate number of cells to be contacted in view of the given route of administration, and it is even more preferred that about 3 to about 300 pfu enter each cell. These viral quantities can be varied according to the need and use whether in vitro or in vivo. The actual dose and schedule can also vary depending on whether the composition is administered in combination with other compositions, e.g., pharmaceutical compositions, or depending on individual differences in pharmacokinetics, drug disposition, and metabolism.

The AAV vectors can be formulated for medical purposes according to standard procedures available in the art, e.g., a pharmaceutically acceptable carrier (or excipient) can be added. A carrier or excipient can be a solid, semi-solid or liquid material which can serve as a vehicle or medium for the active ingredient. The proper form and mode of administration can be selected depending on the particular characteristics of the product selected, the disease, or condition to be treated, the stage of the disease or condition, and other relevant circumstances (Remington's Pharmaceutical Sciences, Mack Publishing Co. (1990)). The proportion and nature of the pharmaceutically acceptable carrier or excipient are determined by the solubility and chemical properties of the substance selected the chosen route of administration, and standard pharmaceutical practice. The growth factors, derivatives thereof, a nucleic acid coding sequence thereof of the present invention, while effective themselves, can be formulated and administered as pharmaceutically acceptable salts, such as acid addition salts or base addition salts, for purposes of stability, convenience of crystallization, increased solubility, and the like.

By “treating” is meant the slowing, interrupting, arresting or stopping of the progression of the disease or condition and does not necessarily require the complete elimination of all disease symptoms and signs. “Preventing” is intended to include the prophylaxis of the neurological disease, wherein “prophylaxis” is understood to be any degree of inhibition of the time of onset or severity of signs or symptoms of the disease or condition, including, but not limited to, the complete prevention of the disease or condition.

The mammal to be treated can be, for example, a guinea pig, dog, cat, rat, mouse, horse, cow, sheep, monkey or chimpanzee. In one embodiment, the mammal is a human. Likewise, in one embodiment G-CSF used for therapy or prophylaxis is a human factor or derived from a human source. In turn, the mammal or subject in need of the treatment described herein is one who is suffering from or expected to develop ALS based on clinical assessment.

The recombinant vectors delivering G-CSF are provided to the spinal cord region and care should be exercised so as to avoid inserting the vector or disrupting the necessary neural connections and function of the spinal cord. For example, delivery to the spinal cord region includes intraspinal injection and in a preferred embodiment, the delivery is systemic administration via intraspinal injection. Intrathekal administration or direct delivery to the motor cortex may also be employed. Preferably, the lumbar or cervical regions of the spinal cord are targeted for delivery. Combinations of different regions can be targeted, simultaneously or at different times.

### EXAMPLES

### Methods

ALS Model

The animals used for the experiments were transgenic for the SOD1(G93A) mutation on a C57BL/6 background (B6.Cg-Tg(SOD1-G93A)1Gur/J strain; Jackson Laboratory, Bar Harbour, Me., USA). They harbour a high copy number of the mutant human SOD1 transgene. For animals with a delay in their onset of disease (no symptom at week 12 of age), the transgene copy number was determined by quantitative PCR to control against drops in copy number that might modify the disease phenotype. The heterozygous line was maintained by mating transgenic males with C57BL/6 wild-type females. Transgenic females were used in all experiments. The animals were age-matched with equally distributed siblings to treatment and control groups.

Recombinant AAV G-CSF Vector

Generation of AAV G-CSF was performed by subcloning the murine G-CSF cDNA sequence into the AAV2 backbone plasmid containing the chicken β-actin promoter and an IRES-eGFP sequence, flanked by AAV2 ITR sequences. AAV-eGFP as control vector was generated by inserting the coding sequence of eGFP into the same AAV expression cassette. HEK293 cells were used for the production of pseudotyped chimeric AAV1/2 vectors (containing a 1:1 ratio of capsid proteins serotype 1 and 2) as described previously (Klugmann et al., (2005), Mol Cell Neurosci 28: 347-360). Cultured cells (80% confluent) propagated in complete DMEM were transfected with the AAV construct and helper plasmids (pH21, pRV1 and pFΔ6) using calcium phopshate. 48 h later, cells were harvested in PBS, centrifuged, and pellets from 5 plates were pooled in 25 mL of a buffer consisting of 150 mM NaCl, 20 mM Tris pH8, 1.25 mL of 10% Natirumdeoxycholate and 50 U/mL of benzonase. After an incubation of 1 hour at 37° C., 25 mL of 150 mL NaCl and 1.25 mL of 10% Natirumdeoxycholate were added and the solution was centrifuged. The supernatant was collected and filtered with 450 mM NaCl, 20 mM Tris pH 8 through a high affinity heparin column (1 mL HiTrap Heparin, Sigma) previously equilibrated with buffer (150 mM NaCl, 20 mM Tris pH 8), at a speed of 1 mL/min as described. The genomic titre of the viral solutions was determined by real-time PCR (light cycler, Roche diagnostics).

Injection of AAV G-CSF

All injections were done in symptomatic 70 day old female mice.

Intramuscular injections. Mice were anesthetized by inhalation of 70% N₂O, 30% O₂, and 1% Halothane. The gastrocnemius and the longissimus thoracis were chosen as target muscles. Injections were done using a nanofil syringe (WPI) and a 33 gauge needle. After incision of the skin at the level of the thoracic spinal column, the longissimus thoracis was exposed. A total of 6 μL of viral solution was injected bilaterally (total of 9×10⁹ AAV particles) in one site per gastrocnemius and two sites per longissimus thoracis (1 μL per site). Intraspinal injections. Mice were anesthetized by injection of a mixture of Ketamine (120 mg/kg body mass, Pharmanovo GmbH) and Xylosine (Rompun, 16 mg/kg body mass, Bayer). Injection anesthesia was chosen instead of inhalational anesthesia here because of better compatibility with the stereotactic procedure. After incision of the skin at the level of the thoracic/lumbar segment of the spinal column, the spinal cord was exposed after sectioning the paraspinous muscles. The tissue between the processi spinosi of T13 and L1 vertebrae was removed. Glass microcapillaries connected to a vacuum pump were used to inject a total of 1 μL of viral solution bilaterally at the L1 level (total of 3.78×10⁹ AAV particles). To prevent any leaking of viral solution, the glass microcapillaries were allowed to remain in place for at least 1 min after each injection, and were retracted slowly from the spinal cord. Curaspon sponge (CuraMedical B.V.) was placed over the injection site, and muscles and skin were sutured. Animals were allowed to wake up and recover from the operation under a heating lamp for one hour. All animal experiments were approved by the Regierungspräsidium Karlsruhe, Germany.

Sciatic Nerve Crush Injury

15 week old mice were anesthetized by inhalation of 70% N₂O, 30% O₂, and 1% Isoflurane. Before any surgical procedures, depth of anesthesia was controlled by a pain reflex elicited by pinching the skin between the toes, and by the palpebral reflex, elicited by touching the eyelids. Under narcosis, hind limbs were shaved and washed with 70% ethanol. The sciatic nerve was exposed at midthigh level and pinched for 30 seconds with fine forceps (S&T JFA-5b, S&T). Skin was sutured (Ethilon USF 4/0) and animals were allowed to recover under a heating lamp for 1 h.

Quantification of G-CSF Expression

After deep anesthesia, blood samples were collected in heparinised tubes after heart puncture, and the spinal cord was dissected after careful transcardial perfusion with Hank's balanced salt solution (HBSS). Entire spinal cords were homogenized in lysis buffer (Promega). G-CSF concentration was measured by an ELISA for mouse G-CSF (Quantikine, RD systems) from the serum after centrifugation, or from the protein extract.

Hematology

After deep anesthesia, blood samples were collected in heparinised tubes after heart puncture. White blood cell count was performed by an automatic cell-counting system (Cell-Dyn 4000™ Hematology analyser, Abbott). The system uses optical flow cytometric technology to obtain the white blood cell count and analyse subpopulations, such as neutrophils.

Assessment of Disease Progression and Survival

One week before vector injection (at 64 days of age) mice were trained for all motor-behaviour exercises. All tests were done weekly. Rotarod sessions lasted 470 s and a constant accelerating mode from 3 to 30 rpm was used (Rotarod, UGO Basile). Between each session, the mice were allowed to rest for 470 seconds. Mean of three tests was recorded. Muscular strength was measured by grip strength measurements (GS Columbus). Mean of three tests was recorded. Weight evolution and clinical symptoms were assessed weekly. Clinical end stage of the disease was defined as the inability of the animal to right itself over a period of 30 seconds. Animals were sacrificed at this point.

Immunohistochemistry

Counting of motoneurons. After deep anaesthesia, mice were transcardially perfused with HBSS followed by 4% paraformaldehyde, spinal cords were dissected and embedded in paraffin. Coronal paraffin sections 10 μm thick from the lumbar or cervical spinal cord were stained for choline acetyltransferase (CHAT) using the avidin-biotin complex (ABC) technique with 3,30-diaminobenzidine hydrochloride as chromogen (DakoCytomation). Nuclei were stained with haemalaun solution. All neurons in the ventral horn that had a clearly identifiable nucleolus, were >400 mm² in size, and were CHAT-positive were counted (see (Pitzer et al., (2008), Brain 131: 3335-3347)). Ten sections per mouse spinal cord that were 100 mm apart over a length of 1 mm isolated from the lumbar spinal cord were counted. Measurements were done on a total of 18 mice from the AAV control (n=9) and AAV G-CSF groups (n=9).

Counting of microglia. Coronal paraffin sections 10 μm thick from the spinal cord were stained for ionized calcium binding adaptor molecule 1 (IBA-1, 19741, WAKO, rabbit, 1:200). All microglia were counted in ten sections per mouse spinal cord that were 100 mm apart over a length of 1 mm isolated from spinal cord were counted. Measurements were done on a total of 12 mice from the AAV-eGFP (n=4), AAV G-CSF SOD-1 (G93A) (n=4) groups and wild type littermates (n=4).

Counting of neuromuscular junctions (NMJs). After deep anaesthesia, mice were transcardially perfused with HBSS followed by 4% paraformaldehyde, muscles were dissected and cryoprotected for 1 h in 30% sucrose solution, frozen on dry ice, and stored at −80° C. 40 μm thick cryosections were stained for presynaptic structures (axons) with α-neurofilament-L (AB9568, Chemicon, rabbit, 1:200) and for the nicotinic acetylcholine receptor (nAChr) with α-bungarotoxin-TRITC (T1175, Invitrogen, 1:200). Five sections per animals were counted, corresponding to ˜200 nAChrs per animal.

Statistics

Experiments were performed in a randomized and blinded manner, including computer-generated probe randomizations and probe labelling, blindness of all experimenters to treatment identities until the end of the experiment, and separation of data analyses from experiment conduction. Animals were age- and littermatched. Group or pairwise parametric or non-parametric comparisons were done using NCSS software (NCSS, Kaysville, Utah, USA) or JMP 8.01 (SAS Institute). Survival and onset data were analysed using the log-rank test. A p-value<0.05 was considered significant.

Results

Muscular Injection Fails to Transduce Motoneurons in SOD-1 (G93A) Transgenic Mice

An elegant and clinically feasible way to bring G-CSF to motoneurons would be to exploit the retrograde transport ability of AAV and inject the virus into skeletal muscles where it would be taken up by presynaptic neuromuscular junctions. Indeed, successful targeting of motoneurons with AAV using this route has been described (Kaspar et al., 2003 Science 301: 839-842). Motoneurons are then expected to synthesize and secrete G-CSF that will bind to its neuronally expressed receptor and induce antiapoptotic pathways. Such a strategy would have the additional advantage of mimicking the endogenous autocrine behaviour of the ligand in neurons (Schneider et al., (2005), J Clin Invest 115: 2083-2098).

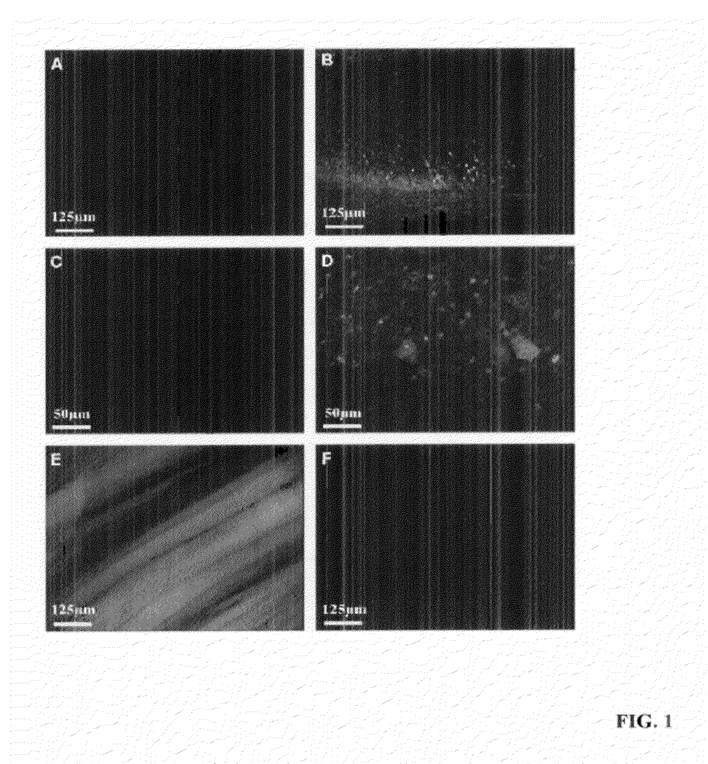

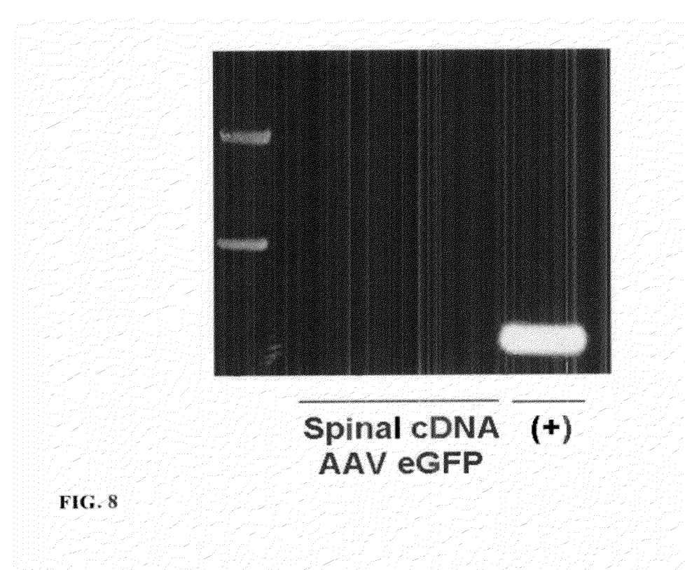

SOD-1 (G93A) transgenic mice were injected with a total of 0.9×10¹⁰ particles of G-CSF-expressing and control virus into the gastrocnemius and longissimus thoracis muscles. And next studied virus-mediated eGFP expression 4 weeks after injection, i.e. the reported time for the maximal expression of AAV-encoded proteins (Palomeque et. al., (2007), Gene Ther 14: 989-997). Surprisingly, we were unable to detect any fluorescent signal in the spinal cord of i.m. injected mice (n=10; FIG. 1a, c), while there was strong eGFP expression in all injected muscles (FIG. 1e), suggesting either that AAV particles were not retrogradely transported or that transported particles did not lead to detectable amounts of eGFP expression. For a more sensitive detection of virus presence in the spinal cord after i.m. delivery, we employed qPCR analysis of DNA extracted from the thoracic and lumbar spinal cord. However, we were unable also with this method to detect viral DNA in the spinal cord of any of the animals studied (n=4; FIG. 8).

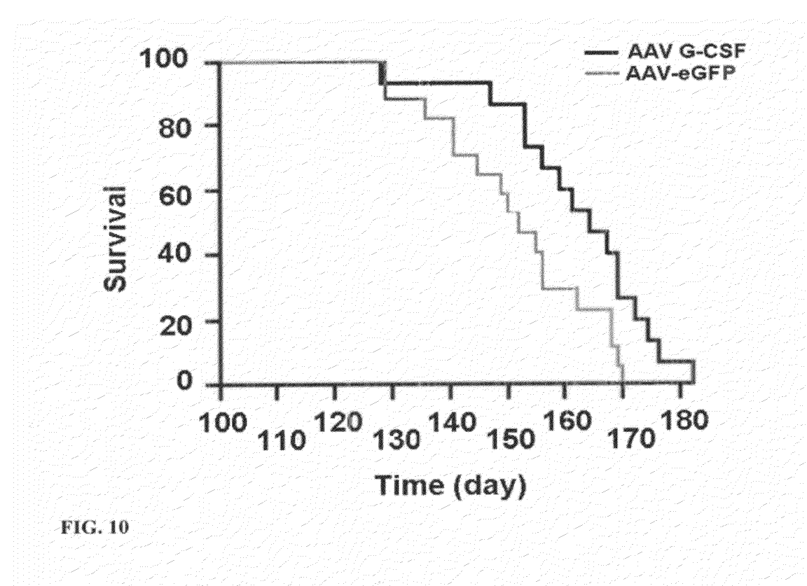

We therefore decided to directly inject viral particles into the spinal cord to transduce motoneurons (Lepore et al., (2007) Brain Res 1185: 256-265). This mode of injection led to strong and mainly neuronal expression of the virus-derived eGFP in the ventral spinal cord (FIG. 1d) (Klugmann et al., (2005), Mol Ther 11: 745-753) over a length of at least 2 mm (FIG. 1b, d), but not in the musculature (FIG. 10. Thus, intramuscular injection of AAV particles in our hands did not result in detectable retrograde transport of the virus, while intraspinal delivery appears as a very efficient way to deliver AAV particles to the spinal cord.

### Intraspinal Injection of AAV Leads to a Highly CNS-Specific Expression of G-CSF

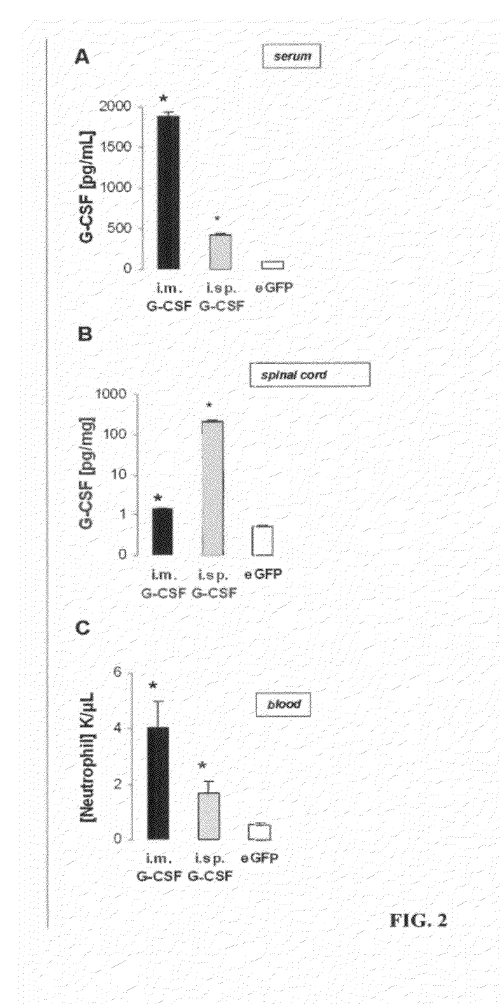

After intramuscular injection at week 15 we observed a high level of G-CSF in the serum accompanied by a moderate increase of G-CSF in spinal cord extracts. In contrast to this, intraspinal injection of AAV GCSF strongly increased spinal G-CSF levels, but only moderately elevated serum G-CSF (serum: i.m. G-CSF: 1890 pg/mL; i.sp. G-CSF: 420.1 pg/mL; eGFP: 92.5 pg/mL; p<0.05 for both injections; spinal cord: i.m. G-CSF: 1.25 pg/mg; i.sp. G-CSF: 229.2 pg/mg; eGFP: 0.3 pg/mg; p<0.05 for both injections FIG. 2a, b)

Serum G-CSF is able to stimulate the proliferation of neutrophil precursors and their differentiation into mature neutrophilic granulocytes (Neidhart et al., (1989) J Clin Oncol 7: 1685-1692). Indeed, we noted a significant elevation of neutrophils after both intramuscular and intraspinal injections when compared to the control SOD-1 (G93A) mice. While neutrophil count was 8-fold elevated with i.m. injection (4.04 neutrophils/nl), intraspinal delivery resulted only in a 3-fold elevation (1.68 neutrophils/nl), still within the normal value range for mice (Hedrich et al., (2004), Elsevier Academic Press: p. 278) (FIG. 2c). Thus, muscular injection of AAV predominantly led to a systemic delivery of G-CSF produced in the injected muscles, along with its expected consequences in terms of neutrophil elevation and did not pose any advantage over systemic subcutaneous delivery. In contrast, intraspinal injection led to a highly CNS-specific delivery with low systemic levels, and a moderate increase of neutrophils, suggesting that this mode of delivery could maximize the neuroprotective effects, and minimize the peripheral effects. We thus focused our efforts on this mode of delivery.

Spinal Delivery of G-CSF is Beneficial for SOD-1 (G93A) Mice

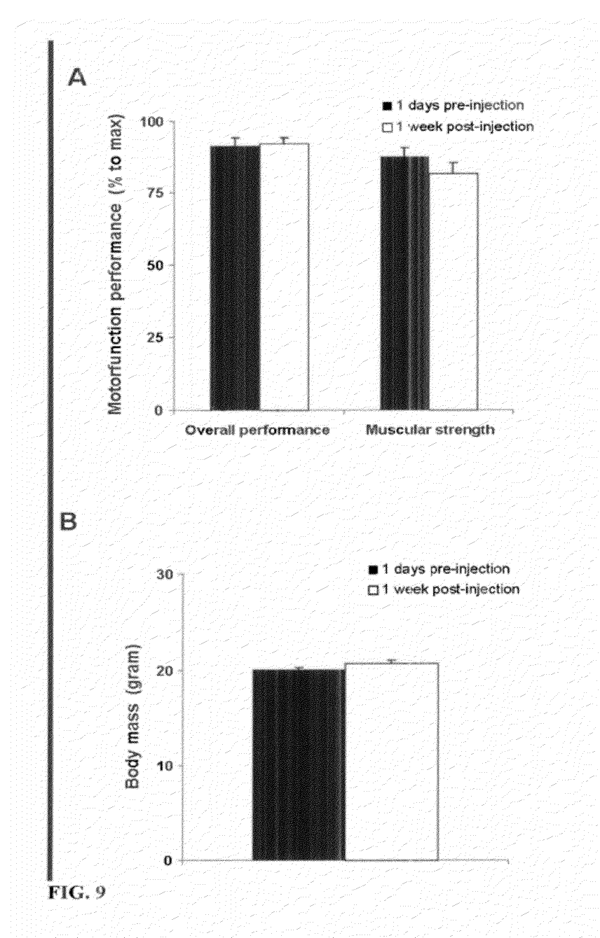

Since intraspinal injection in contrast to i.m. injection requires a relatively lengthy surgery, we studied post-operative behaviour, weight, and motor performance of the mice and found no obvious difference between mice before and one week after surgery (FIG. 9), suggesting that the surgery did not have a major impact on general mouse health and motor functions.

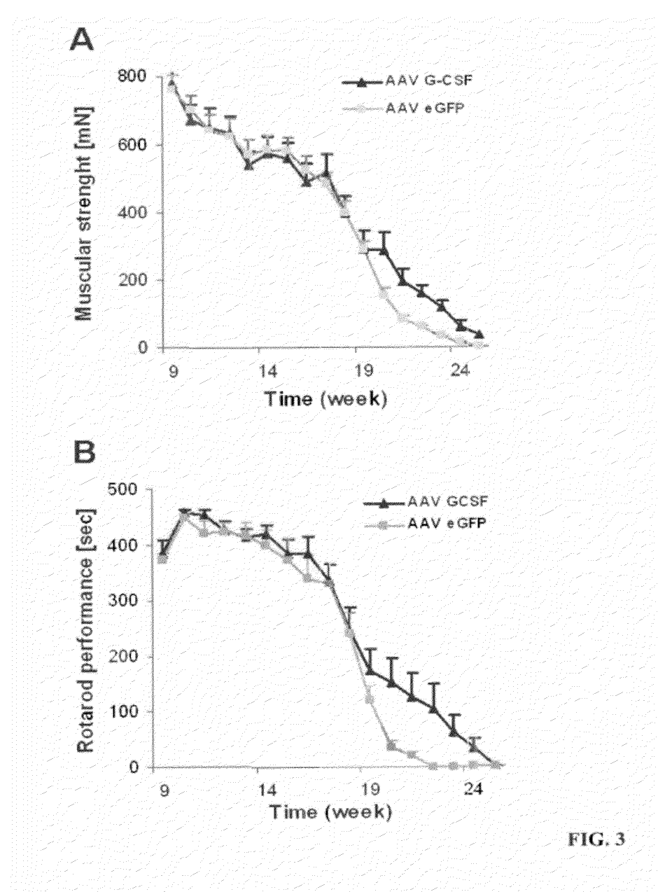

We monitored Rotarod and grip strength performance as indicators of muscular endurance and strength weekly in AAV G-CSF or control injected animals (n=12 female mice per group, littermatched). Our experimental settings comply with the guidelines for preclinical studies in ALS established by the European ALS/MND group (Ludolph et al., (2010) Elsevier Academic Press: p. 278). From week 20 on we noted a relative improvement in muscular strength in the G-CSF versus the control group (AAV eGFP: 151 mN; AAV G-CSF: 285 mN; p<0.05 by repeated measures ANOVA and Fisher's LSD) and a better performance on the rotarod (AAV eGFP: 34 sec; AAV G-CSF: 135 sec, p<0.05 by repeated measures ANOVA and Fisher's LSD) (FIG. 3).

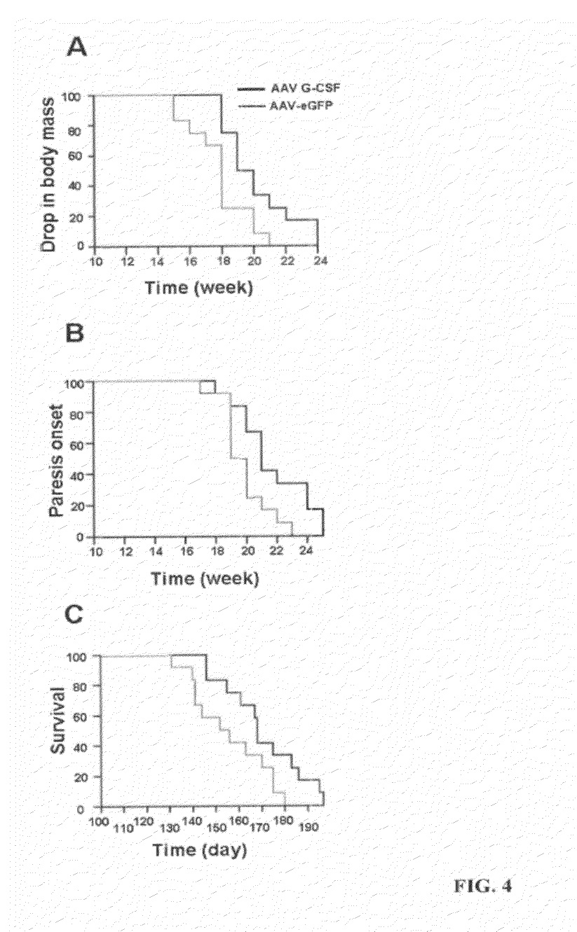

The disease progression in SOD-1 (G93A) mice is well described. The disease at its onset is characterized by a slight hind limb tremor, the midpoint of disease is defined by gait impairment and weight loss, and the endstage of disease is marked by paresis. Onset of body mass decrease, defined as a drop of 5% of the mouse maximal weight (around 1 gram), was significantly delayed by G-CSF treatment by more than 2 weeks (p<0.05; FIG. 4a). The onset of gait impairment, defined as abnormal limb movement in at least one hind limb, was not significantly different between the two groups despite a trend (p<0.17), but the onset of paresis, defined as the inability to use one limb in the coordinated stride, was significantly delayed after G-CSF treatment (p<0.05; FIG. 4b). Most importantly, the clinical end point of the disease was delayed by 15 days, increasing the survival by 10% (FIG. 4c). This gain in survival was higher than the increases observed after subcutaneous delivery (7% increased survival (Pitzer et al., (2008), Brain 131: 3335-3347)). An increase of 7% in survival was also seen in the mice treated by i.m. injection, which was essentially a systemic delivery (FIG. 10). Thus, G-CSF delivered by AAV to the spinal cord is able to delay disease progression and improve survival in SOD-1 (G93A) mice.

Spinal Delivery of G-CSF Maintains Motor-Unit Integrity in SOD-1 (G93A) Tg Mice

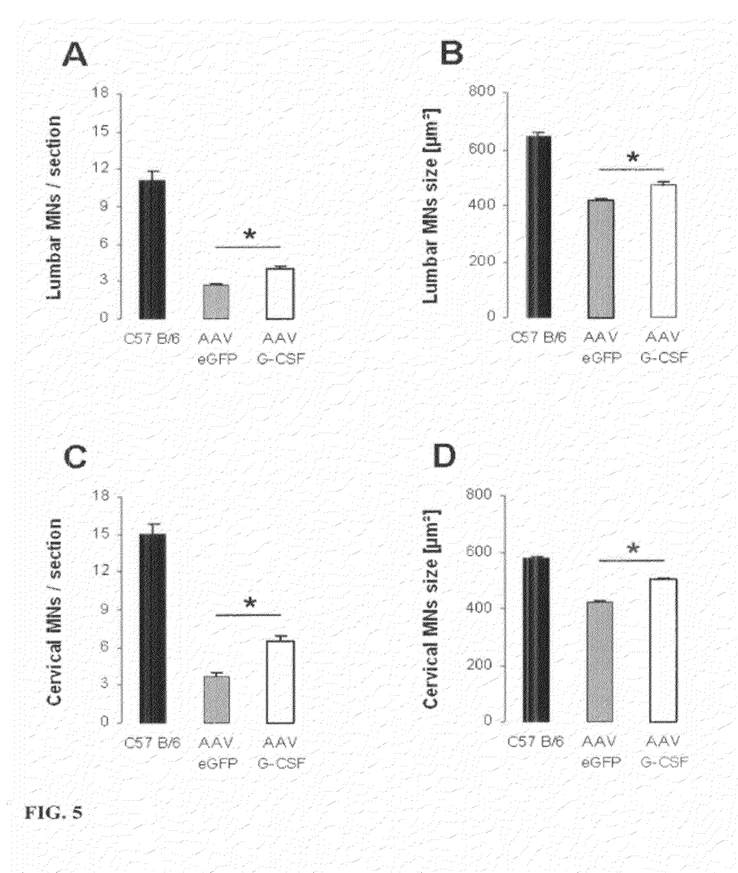

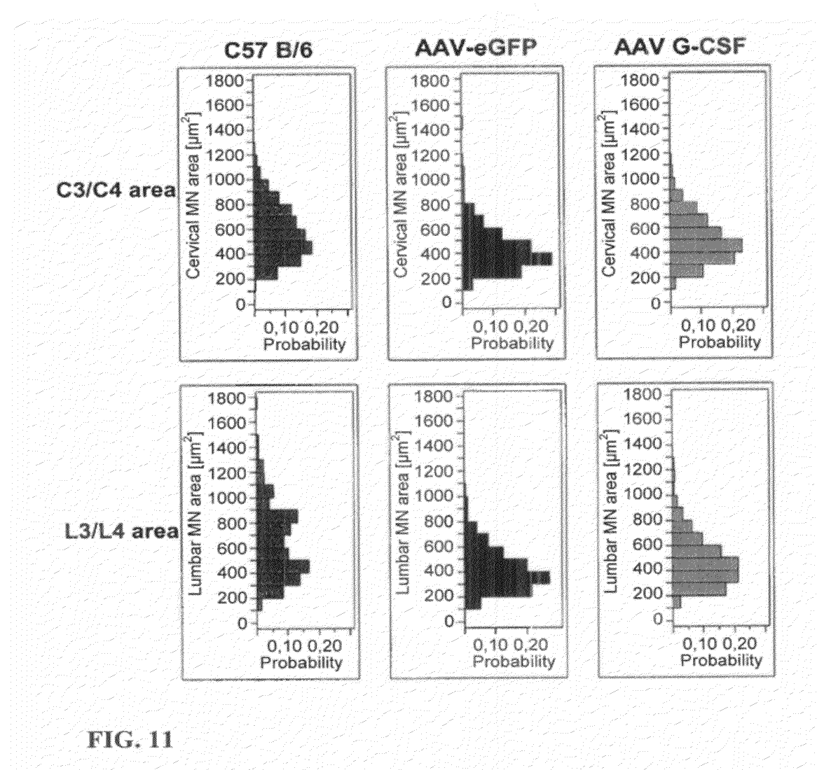

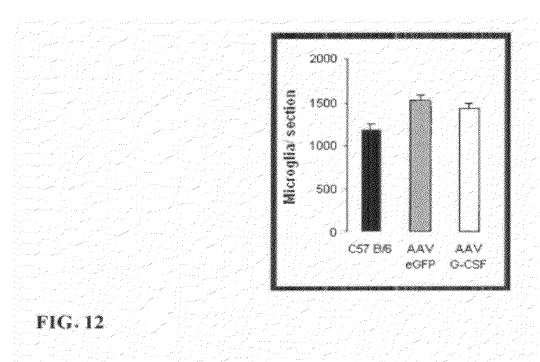

Motoneuron survival. G-CSF is known to protect α-motoneurons under pro-apoptotic conditions (Henriques et. al., (2010), BMC Neurosci 11:25; Pitzer et al., (2008), Brain 131: 3335-3347). We have previously shown an increase by 30% in the total number of α-motoneurons at midpoint of the disease (week 15) after systemic delivery of G-CSF (Pitzer et al., (2008), Brain 131: 3335-3347. Here, we sought to determine the effect of a direct delivery of G-CSF to motor neurons at two different spinal segments: the cervical (C3-C4) and lumbar level (L3-L4) of 15 weeks old SOD-1 (G93A) mice. We used the previously described criteria based on localisation, size and ChAT positivity (Pitzer et al., (2008), Brain 131: 3335-3347). We noted a loss of α-motoneurons at both lumbar and cervical spinal segments for the SOD-1 (G93A) mice when compared to the littermate wild types (p<0.05; FIG. 5). After G-CSF treatment, we noted a rescue of α-motoneurons at both the cervical (+50% motoneurons; p<0.05; FIG. 5a) and lumbar level (+35% motoneurons; p<0.05; FIG. 5c). The analysis of the size distribution of the remaining motoneurons indicates that large α-motoneurons are particularly protected by G-CSF treatment at both levels (FIG. 5b, d; p<0.05). FIG. 11 shows a histogram size distribution of cervical and lumbar motoneurons. In addition we assayed microglial numbers in the spinal cord as a possible cellular element contributing to disease pathophysiology (Boillee et al., (2006) Science 312: 1389-1392). While microglial numbers were increased in the SOD-1 (G93A) model at week 15 in contrast to wt littermates, we could not detect any influence of G-CSF on this elevation, a result in concordance with our previous study (Pitzer et al., (2008), Brain 131: 3335-3347) (FIG. 12).

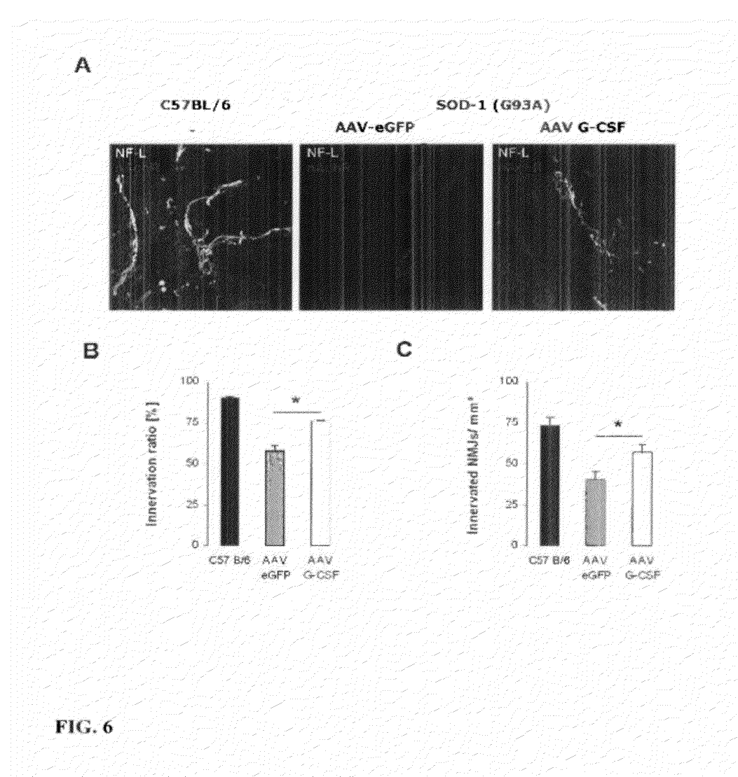

Preservation of neuromuscular junctions. In ALS, the disruption of the neuromuscular junctions (NMJs) occurs long before the degeneration of the cell body of motoneurons (Fischer et al., (2004), Exp Neurol 185: 232-240). Therefore, the rescue of α-motoneuron cell bodies is not sufficient to explain a therapeutic effect that inherently implies preserved muscle innervation (Dupuis et. al., (2009), Curr Opin Pharmacol 9: 341-346). To determine whether G-CSF treatment can preserve muscular innervation, we investigated the state of the NMJs in the gastrocnemius muscle of 15 week old mice. At this age, mice present clear gait impairment and decreased performance in both rotarod and grip strength analyses. At first, we determined the innervation fraction of the gastrocnemius muscle, defined as the number of innervated NMJs per total NMJs (innervated and denervated). We found that the gastrocnemius muscle in SOD1 (G93A) mice showed clear denervation when compared to the littermate control wild type mice where virtually all muscular endplates were found innervated (FIG. 6a). The severity of this denervation is reduced by G-CSF treatment (AAV eGFP: 57.7% innervation ratio; AAV G-CSF: 75.7%; wild type: 90.2%; p<0.05; FIG. 6b). This effect is also seen when comparing the total number of innervated NMJs per muscle volume (40% increase in the total number of NMJs after AAV G-CSF transduction; AAV eGFP: 40.6 innervated NMJs/mm³; AAV G-CSF: 57.4; p<0.01; FIG. 6c).

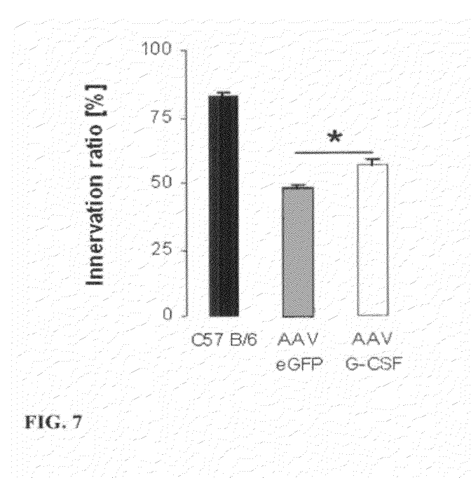

Motor axon regeneration. An important intrinsic compensatory mechanism in ALS is that surviving motoneurons partially re-innervate postsynaptic NMJ sites that belonged to the motor unit of a damaged neighbouring motoneuron (Schaefer et al., (2005) J Comp Neurol 490: 209-219). A G-CSF-induced higher propensity for motor axon outgrowth may therefore be an additional mechanism that leads to a higher number of innervated NMJs, especially since G-CSF enhances neurite outgrowth in vitro (Pan et al., (2009), Biochem Biophys Res Commun 382: 177-182; Pitzer et al., (2010), J Neurochem). To approach this question, we performed sciatic nerve crush injury on SOD-1 (G93A) mice at 15 weeks of age. Sciatic nerve crush in adult mice results in axonal degeneration and in muscular denervation. Due to the small length of the sciatic nerve and a high regenerative potential in mice, regeneration usually occurs fast and is complete within 2 weeks in wild type mice (Griffin et al., (2010), Exp Neurol 223: 60-71). Six days after nerve crush we counted the percentage of innervated neuromuscular junctions in the gastrocnemius muscle, ipsilateral to the nerve injury. Reinnervation was almost complete in wild type mice after 6 days, whereas for the SOD-1 (G93A) mice it was strongly impaired (p<0.005) possibly due to axonal transport disturbances (Warita et al., (1999), Brain Res 819: 120-131). AAV-mediated G-CSF treatment led to a higher reinnervation rate in the SOD-1 (G93A) mice (AAV eGFP: 48.0%; AAV G-CSF: 56.4%; wild type: 82.3%; p<0.005; FIG. 7).

In conclusion, intraspinal delivery of G-CSF was able to potently preserve NMJs and stimulate axonal regeneration.

### Discussion

The results of this study demonstrate that intraspinal delivery of G-CSF through viral gene therapy improves treatment effects of this neurotrophic protein, while minimizing unwanted systemic effects. These data also further solidify our chain of arguments for a direct motoneuronal mode-of-action of G-CSF versus indirect effects mediated by its hematopoietic effects. Our results also suggest that the chimeric AAV1/2 serotype employed here is not a highly efficient means for retrograde transport to motor neurons even if this serotype is better at transducing neurons than AAV2 alone.

Retrograde AAV Delivery by Intramuscular Injections?

We could not detect any retrograde transport of AAV after intramuscular injection, both measured by EGFP expression and PCR, and confirmed by the distribution profile of G-CSF. This is unexpected, and at odds with several published studies reporting successful retrograde transduction of motoneurons (Hollis et al., (2008), Mol Ther 16: 296-301; Kaspar et al., (2003) Science 301: 839-842). The amount of virus used has been comparable between our work and published studies (Hollis et al., (2008), Mol Ther 16: 296-301; Kaspar et al., (2003) Science 301: 839-842).

One possible reason for the discrepancy to published reports may lie in the AAV serotype used. Originally, retrograde transduction after muscle injection has been demonstrated for serotype 2 (Kaspar et al., (2002), Mol Ther 5: 50-56; Kaspar et al., (2003) Science 301: 839-842). In a systematic comparison of retrograde transduction efficiency of different (self-complimentary) serotypes AAV1 performed much better than AAV2 which did not result in detectable spinal cord transduction after i.m. injection (Hollis et al., (2008), Mol Ther 16: 296-301). We have used the chimeric rAAV 1/2 because of its superior transduction efficiency and neuronal preference in contrast to AAV2 (Klugmann et al., (2005), Mol Ther 11: 745-753; Klugmann et al., (2005) Mol Cell Neurosci 28: 347-360) matching the transduction efficiency and tropism of AAV1 reported by others (Passini et al., (2003), J Virol 77: 7034-7040; Taymans et al., (2007), Hum Gene Ther 18: 195-206). AAV1/2 was also reported to be trans-synaptically transported in the nigrostriatal pathway (Franich et al., (2008) Mol Ther 16: 947-956). Our failure to transduce neurons is therefore not easily explained by the chosen serotype.

We believe that the most likely explanation for our failure to detect any retrograde transport is the very low retrograde transduction efficiency resulting in a borderline success rate of the process. In addition to the above mentioned data from Hollis with AAV2, at least one other group has been unable to detect retrograde transduction after i.m. injection (Li et. al., (2006), Toxicol Appl Pharmacol 214: 152-165). Novel self-complimentary viruses and partial nerve demyelination appear to produce higher transduction efficiencies (Hollis et al., (2008), Mol Ther 16: 296-301; Hollis et al., 2010). Overall, the low efficiency of this application mode makes it unsuited for any clinical considerations.

Despite the failure of direct CNS transduction we found G-CSF elevated in the spinal cord since G-CSF is able to cross the BBB. Predictably, this also leads to a therapeutic effect on survival similar to systemic subcutaneous pump delivery (7% increase in survival) (Pitzer et (2008), Brain 131: 3335-3347). At the same time, constant and massive production of the virally expressed G-CSF from muscle led to high serum concentrations and clear elevations of WBC count. This would generate a considerable safety risk to patients as the virus cannot be shut off. Most of the time, quantification and distribution of the synthesized proteins are not indicated in studies with AAV (Kaspar et al., (2003) Science 301: 839-842; Dodge et al., (2008), Mol Ther 16: 1056-1064; Lepore et al., (2007) Brain Res 1185: 256-265), making the comparison between our results with previous works difficult with regard to relative CNS specificity of delivery. Our results conclusively show that intramuscular injection of AAV is not a feasible route for CNS-targeted therapy with G-CSF.

Intraspinal Injections have a Favourable Efficacy and Specificity Profile

Injection into the spinal cord led to a rather specific CNS-delivery. Elevation of G-CSF concentration in the serum was moderate (about 3-fold) and the elevation of WBCs still in the normal range for mice (Hedrich et. al., (2004) Elsevier Academic Press: p. 278). The peripheral load of G-CSF was still more than 2-fold lower after intraspinal injection when normalizing for the 2.4-fold lower total virus load injected intraspinally versus i.m. At the same time, CNS levels of G-CSF were 200-fold higher, generating a very favourable specificity profile for this delivery mode. It is unclear at present whether the systemic elevation of G-CSF after intraspinal delivery originates from spurious transduction of muscles (e.g. in the injection canal), or from leakage or secretion of intrathecally produced G-CSF into the blood stream.

Although only injected at the lumbar level, motoneuron protection was also seen in the cervical spinal cord, presumably because of sufficient distribution of the secreted protein within the spinal extracellular space. Injections were made at week 10, likely resulting in relevant protein expression at week 12-13 (no expression seen 1 week after delivery, data not shown), and resulted in clear benefits on motor function and survival. Although expression of G-CSF and therefore therapy started in the symptomatic phase, approximately 2 weeks later than done previously (Pitzer et al., (2008), Brain 131: 3335-3347), survival was increased from 7 to 10% by specific CNS delivery. The higher survival after a highly specific CNS delivery suggests that the benefit after G-CSF treatment is caused by its direct neuroprotective activity rather than a stimulation of the hematopoietic system or influences on microglia. Thus, direct intraspinal injection of AAV appears as a preferred approach for G-CSF delivery with a minimum of systemic side effects.

Effect Size and Translatability of Findings in the Mouse Model

Do the effects seen in this mouse model justify clinical testing of the G-CSF concept in patients suffering from ALS? We have seen strong beneficial effects on motor function, and increases in survival in the range of 7-10% using various application modes of G-CSF. It is however fully unclear at present how an increase in survival seen in the mouse may translate to the human. Translated linearly, a prolongation of life expectancy of 10% in human patients would certainly be a clinically highly meaningful benefit (˜6 yrs).

The real issue with therapeutic experiments in the SOD-1 mouse is however the low reliability of animal efficacy data due to insufficient rigorousness (blinding, randomization, SOPs, sample size, control for confounding factors) in the conduction of the animal studies (Scott et al., (2008) Amyotroph Lateral Scler 9: 4-15). Indeed, most of the positive results reported in the literature cannot be reproduced under conditions of standardized testing. Our studies have been conducted under highly standardized conditions including rigorous blinding and randomization procedures, and have been reproduced in multiple studies with different application modes. Since G-CSF is also the first growth factor which appears clinically feasible in terms of safety and pharmacokinetics we believe that this approach is worthwhile to be tested in human patients.

Clinical Relevance of AAV Therapy for ALS

AAV vectors, particularly AAV2, have been evaluated in clinical trials in a considerable number of diseases, among those Parkinson's disease (Kaplitt et. al., (2007), Lancet 369: 2097-2105), cystic fibrosis (Moss et. al., (2007) Hum Gene Ther 18: 726-732), muscular dystrophy (Rodino-Klapac et al., (2008) Neurology 71: 240-247), Alzheimer's disease (Mandel et al., (2010) Curr Opin Mol Ther 12: 240-247), Leber's congenital amaurosis (Maguire et al., (2008) N Engl J Med 358: 2240-2248), and hemophilia B (Manno et al., 2006 Nat Med 12: 342-347). Although the total number of patients treated is still small, AAV therapy has not presented major safety issues yet. Findings of liver carcinogenesis in neonatal mice are likely an isolated finding (Kay M A, (2007), Nat Biotechnol 25: 1111-1113). AAV therapy for ALS patients appears attractive, even if it has to be done by intraspinal injections. The main problem of chronic G-CSF therapy, increased neutrophil counts, would be avoided by this approach if the findings in mouse can be translated to human patients. A caveat in this approach is that intrathecal G-CSF production cannot be easily turned off in case of any CNS-specific problems. The virus can synthesise therapeutic proteins for a long time period after only one injection, e.g. up to 2 years in non-human primates (Buie et al., (2009), Invest Ophthalmol Vis Sci 51: 236-248). After monkey studies, an initial safety trial using regulatable promoters, for instance the tet-off system, could answer the question if there are safety issues of long-term G-CSF delivery to the CNS (Kordower et al., (2008), Exp Neurol 209: 34-40).

Recently, novel reports have suggested transduction of the CNS via intravenous delivery of the AAV serotype 9 (Duque et. al., (2009), Mol Ther 17: 1187-1196). This appears as a novel interesting delivery route, and needs to be tested in ALS models.

Mechanism of Action of G-CSF: Motor-Unit Preservation

Besides improving the survival of motoneurons, G-CSF treatment preserves neuromuscular junctions in SOD-1 (G93A) Tg mice. Axonopathy and loss of neuromuscular junctions is known to occur in ALS long before motor neuron degeneration and initiation of symptoms (Fischer et al., (2004), Exp Neurol 185: 232-240; Pun et al., (2006), Nat Neurosci 9: 408-419). Many NMJs are lost in the SOD-1 G93A mice from P50 on, before detectable loss of motor axons in the ventral roots exiting the spinal cord, and long before the first symptoms of paralysis. Preservation of NMJs by G-CSF treatment may therefore constitute a complementary protective mechanism, independent of antiapoptotic protection of the α-motoneurons.

The higher innervation rate of the NMJs under G-CSF treatment may be caused by stabilization of the NMJs, by a higher reinnervation of depleted postsynaptic sites, or by both. The present experiment does not allow us to distinguish between these possibilities. Likely the effect seen is a combination of these mechanisms.

A very recent paper claims that subcutaneously applied G-CSF elevates microglial numbers in the spinal cord of SOD1(G93A) transgenic animals, and suggests this as a mechanism of action for G-CSF (Yamasaki et. al., (2010), J Neuroimmunol). We have not observed any alterations of microglial numbers after intraspinal delivery of G-CSF, consistent with earlier work on systemic delivery of G-CSF in ALS models (Pitzer et al., (2008), Brain 131: 3335-3347). Although a number of differences exist between Yamasaki et al. and our studies (glycosylated vs. non-glycosylated G-CSF, continuous versus once daily delivery, different doses of G-CSF used), none of those appears fundamental enough to offer a clear explanation of this discrepancy at present. From a general perspective it appears however rather unlikely that elevation of SOD1-transgenic microglia would make a major contribution to the beneficial G-CSF effects seen in ALS models (Boillee et al., (2006), Science 312: 1389-1392; Gowing et. al., (2008), J Neurosci 28: 10234-10244).

In conclusion, the data shown here further support our concept that the direct action of G-CSF on motoneurons is the major mode-of-action responsible for its beneficial effects in the SOD-1 (G93A) model (Henriques et. al., (2010), BMC Neurosci 11: 25; Pitzer et al., (2008), Brain 131: 3335-3347; Schneider et al., (2005), J Clin Invest 115: 2083-2098).

1. A method of treating amyotrophic lateral sclerosis (ALS) in a mammalian subject in need thereof, the method comprising delivering to a spinal cord region of the mammalian subject, a recombinant vector comprising at least two adeno-associated virus (AAV) inverted terminal repeats (ITRs) flanking a polynucleotide encoding mammalian G-CSF operably linked to a transcriptional promoter that can express the polynucleotide.

2. The method of claim 1, wherein the at least two AAV ITRs are AAV serotype 1 ITRs.

3. The method of claim 1, wherein the at least two AAV ITRs are AAV serotype 2 ITRs.

4. The method of claim 1, wherein the recombinant vector comprises a pseudotype AAV that comprises a portion of one serotype of AAV and a portion of a second, different serotype of AAV.

5. The method of claim 4, wherein one serotype of AAV is AAV-1 and the second, different serotype of AAV is AAV-2.

6. The method of claim 1, the mammalian subject is a human subject.

7. The method of claim 1, wherein the spinal cord region is a lumbar region or a cervical region.

8. The method of claim 1, wherein the polynucleotide encodes human G-CSF.

9. The method of claim 1, wherein the polynucleotide encodes a protein having at least 90% homology to SEQ ID NO:1, a protein having at least 90% homology to SEQ ID NO:2, a protein having at least 90% homology to SEQ ID NO:3, PEG-modified G-CSF or a combination thereof.

10. The method of claim 1, wherein the delivering comprises an intraspinal injection.

11. The method of claim 10, wherein the delivering comprises systemic intraspinal injection.


SEQUENCE LISTING (9 sequences)

SEQ ID NO: 1 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 208 aa, Mus musculus
MAQLSAQRRMKLMALQLLLWQSALWSGREAVPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLA

SEQ ID NO: 3 - protein, 214 aa, Rattus rattus
MKLMALQLLLWHSALWSGQEAIPLLTVSSLPPSLPLPRSFLLKSLEQVRKIQARNTELLEQLCATYKLCHPEELVLFGHSLGIPKASLSSCSSQALQQTKCLSQLHSGLFLYQGLLQALAGISSELAPTLDMLHLDVDNFATTIWQQMESLGVAPTVQPTQSTMPIFTSAFQRRAGGVLVTSYLQSFLETAHHALHHLPRPAQKHFPESLFISI

SEQ ID NO: 4 - protein, 194 aa, Felis catus
KLMALQLLLWHSALWMVQEATPLGPTSSLPQSFLLKCLEQVRKVQADGTALQERLCAAHKLCHPEELVLLGHALGIPQAPLSSCSSQALQLTGCLRQLHSGLFLYQGLLQALAGISPELAPTLDMLQLDITDFAINIWQQMEDVGMAPAVPPTQGTMPTFTSAFQRRAGGTLVASNLQSFLEVAYRALRHFTKP

SEQ ID NO: 5 - protein, 195 aa, Bos taurus
MKLMVLQLLLWHSALWTVHEATPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPTQGAMPTFTSAFQRRAGGVLVASQLHRFLELAYRGLRYLAEP

SEQ ID NO: 6 - protein, 195 aa, Sus scrofa
MKLMALQLLLWHIALWMVPEAAPLSPASSLPQSFLLKCLEQVRKIQADGAELQERLCATHKLCHPQELVLLGHSLGLPQASLSSCSSQALQLTGCLNQLHGGLVLYQGLLQALAGISPELAPALDILQLDVTDLATNIWLQMEDLRMAPASLPTQGTVPTFTSAFQRRAGGVLVVSQLQSFLELAYRVLRYLAEP

SEQ ID NO: 7 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 8 - protein, 175 aa, Homo sapiens
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 9 - protein, 7 aa, Artificial Sequence
  synthetic peptide
LGHSLGI